(19) United States
(12) Patent Application Publication
(10) Pub. No.: US 2018/0003595 A1
(43) Pub. Date: Jan. 4, 2018

(54) SEALED WATER SAMPLING DEVICE

(71) Applicants: Karl Veggerby, Bainbridge Island, WA (US); Ove Veggerby, Bainbridge Island, WA (US)

(72) Inventors: Karl Veggerby, Bainbridge Island, WA (US); Ove Veggerby, Bainbridge Island, WA (US)

(21) Appl. No.: 15/361,472

(22) Filed: Nov. 27, 2016

Related U.S. Application Data
(60) Provisional application No. 62/356,460, filed on Jun. 29, 2016.

(51) Int. Cl. G01N 1/12; G01N 33/18

(52) U.S. Cl. CPC G01N 1/12; G01N 33/1886

(57) ABSTRACT

A sealed cylindrical-type frame with an O-ring sealed lid, and an opening mechanism sealed with O-rings and tension from a coiled spring for water sampling, that may be lowered to a predetermined depth of water without contamination from travel through other depths of water, and may be raised from depths of water without contamination from travel through other depths of water and from travel through air.

### CROSS-REFERENCE TO RELATED APPLICATIONS

This application claims the benefit of, and priority to, U.S. provisional patent application Ser. No. 62/356,460, titled “SEALED WATER SAMPLING DEVICE” and filed on Jun. 29, 2016, the entire specification of which is incorporated herein by reference in its entirety.

### BACKGROUND OF THE INVENTION

### Field of the Art

The disclosure relates to the field of water sampling devices, and more particularly to a sealed water sampling device that is suspended in water from a rod to preserve the integrity of the water sample from contamination.

### Discussion of the State of the Art

In the field of water sampling devices, typical sampling devices have at least one opening that often remains open to the air as well as to the water being sampled. Generally, these devices are lowered to a predetermined depth allowing water from all depths above the desired depth to enter the water sampling device; mixing the desired sample with other depths of water, therefore contaminating the desired water sample. When these open water sampling devices are raised out of the water the sample is then exposed to air when transferring to a separate container causing more contamination of the desired water sample.

Typically, water sampling devices for greater depths (generally depths exceeding fifteen to twenty feet) are more sophisticated and large in size resulting in high costs for devices, as well as the complicating use of such devices; often needing a crane for use of device due to devices size and weight.

What is needed is a means to provide a water sample device that collects from a predetermined depth (generally a depth of zero to twenty feet) in a sealed sampling device, is simple to use and designed to eliminate contamination from other depths of water and contamination from air upon retrieval of the water sample.

### SUMMARY OF THE INVENTION

Accordingly, the inventor has conceived and reduced to practice, in a preferred embodiment of the invention, an apparatus for a sealed water sampling device, that may be lowered to a predetermined depth of water without contamination from travel through other depths of water, and may be raised from a predetermined depth of water without contamination from travel through other depths of water and from travel through air.

In a preferred embodiment of the invention, a cylindrical-type frame made from a solid material with a narrow mouth and lip where water may enter, with a spring sealed lid to prevent contamination.

### BRIEF DESCRIPTION OF THE DRAWING FIGURES

The accompanying drawings illustrate several embodiments of the invention and, together with the description, serve to explain the principles of the invention according to the embodiments. It will be appreciated by one skilled in the art that the particular embodiments illustrated in the drawings are merely exemplary, and are not to be considered as limiting of the scope of the invention or the claims herein in any way.

FIG. 1 is an illustration of an exemplary sealed water sampling device according to a preferred embodiment of the invention, illustrating all components of a water sampling device that is sealed.

FIG. 2 is an illustration of an exemplary water sampling device opened according to a preferred embodiment of the invention, illustrating the method by which a water sampling device may be opened to obtain a water sample and consequently sealed after a water sample is obtained, thereby preventing contamination of the water sample.

FIG. 3 is an exemplary method diagram according to a preferred embodiment of the invention, illustrating the use of a water sampling device.

FIG. 4 is an illustration of an exemplary water sampling device mechanisms, according to a preferred embodiment of the invention, illustrating all components of water sampling device opening mechanisms.

FIG. 5 is an illustration of an exemplary counterweight, according to a preferred embodiment of the invention, illustrating an alternative method for lowering water sampling device without an extension rod.

FIG. 6 is an illustration of an exemplary sealed water sampling device, according to a preferred embodiment of the invention, illustrating a three-quarter angle perspective of water sampling device.

FIG. 7 is an illustration of an exemplary open water sampling device, according to a preferred embodiment of the invention, illustrating a three-quarter angle perspective of water sampling device.

### DETAILED DESCRIPTION

### Detailed Description of Exemplary Embodiments

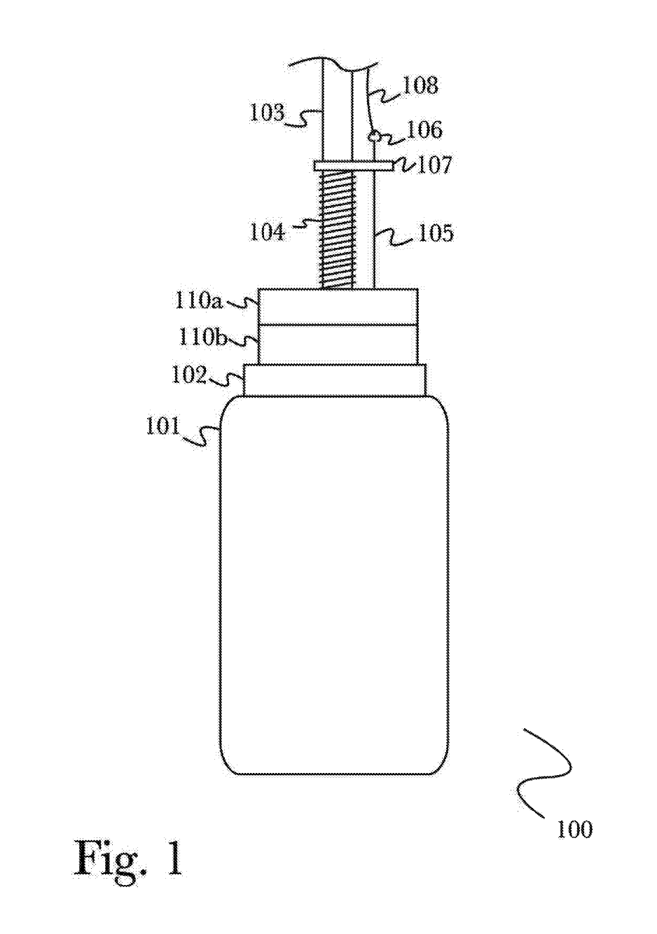

FIG. 1 is an illustration of several exemplary sealed water sampling device 100 according to a preferred embodiment of the invention, illustrating all components 101-108 of a water sampling device that is sealed.

According to the embodiment, a sealed water sampling device 100 may comprise a generally cylindrical-type frame having solid or translucent walls 101 formed of an impermeable material such as metal or plastic, with a narrowing down on one end to form an opening or mouth where an airtight lid 102 with threading may screw onto the device opening or mouth (it should be appreciated that other forms of water sampling devices may be used such as but not limited to, a cylindrical-type frame that does not narrow down to an opening, thereby the opening of the device is the same width as the rest of the device, and other types as well). An airtight lid 102 will generally be comprised of the same or similar impermeable material as water sampling device 101 and will consist of the same width as sampling device 101 opening or mouth (generally device lid 102 is similar or identical to standard collection devices to ease exchange of devices once sample has been collected, however it should be appreciated that other lid types may be used on a preferred use basis) so as to fit over it thereby sealing water sampling device 101 from outside sources, preventing contamination of contents contained within water sampling device 101. A device connecting rod 103 comprised of either metal or a heavy-duty plastic with threads 406 on one end, runs through the center of a disk platform 107, device opening mechanism 110a, 110b, and device lid 102. Device connecting rod 103 is secured by threads 406 to device lid 102 with an acorn nut 407. Attached to device lid 102 by O-rings 203a-n is device opening mechanism 110b. Device opening mechanism 110b has a hollowed out canal 401 where water 202 may enter into water sampling device 101. Attached to device opening mechanism 110b by O-rings 203a-n is device opening mechanism 110a. A coiled spring 104 is wound around device connecting rod 103 between sampling device opening mechanism 110a and device disk platform 107. A lifting rod 105 runs through disk platform 107 and attaches to opening mechanism 110a with a pin 403a-n to secure it in place. Attached to lifting rod 105 is an eye hook 106 at the top for a wire or heavy-duty string 108 (such as but not limited to nylon or other materials) to attach to hook 106 and thereby attaching to lifting rod 105.

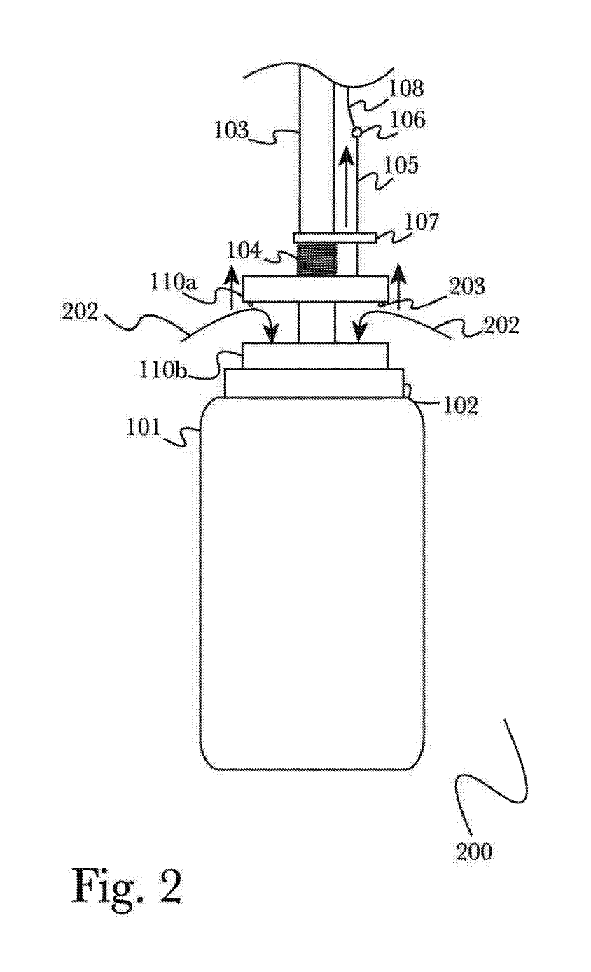

FIG. 2 is an illustration of an exemplary water sampling device 101 opened according to a preferred embodiment of the invention, illustrating the method 200 by which a water sampling device may be opened to obtain a water sample and consequently sealed after a water sample is obtained, thereby preventing contamination of the water sample. Attached to device connecting rod 103 is a rod adaptor 405 that attaches by threads 404 onto a lowering rod so that sealed water sampling device 101 may be lowered to a predetermined depth of water. A wire or heavy-duty string 108 runs alongside lowering rod and rod adaptor 405 and attaches to an eye hook 106 at the top of a lifting rod 105. Lifting rod 105 runs through a disk platform 107 and attaches to opening mechanism 110a with a pin 403a-n. Disk platform 107 is attached to device connecting rod 103 by pin 403a-n. Once wire or heavy-duty string 108 is pulled to capacity, lifting rod 105 lifts through disk platform 107 raising opening mechanism 110a against a coiled spring 104. Once opening mechanism 110a is raised, water 202 can enter water sampling device 101 through opening mechanism 110b canal at the predetermined depth. Once sample 202 is obtained, wire 108 is released lowering lifting rod 105 back through disk platform 107 releasing tension on coiled spring 104, and lowering opening mechanism 110a back onto opening mechanism 110b, forming an airtight seal with the use of O-rings 203a-n. Once device 101 is sealed it is then raised back up on the lowering rod through depths of water without contamination from other depths. Once device 101 has reached top of lowering rod, rod adaptor 405 is detached by threads 404 from lowering rod and sample 202 may be taken sealed in device 101 to study.

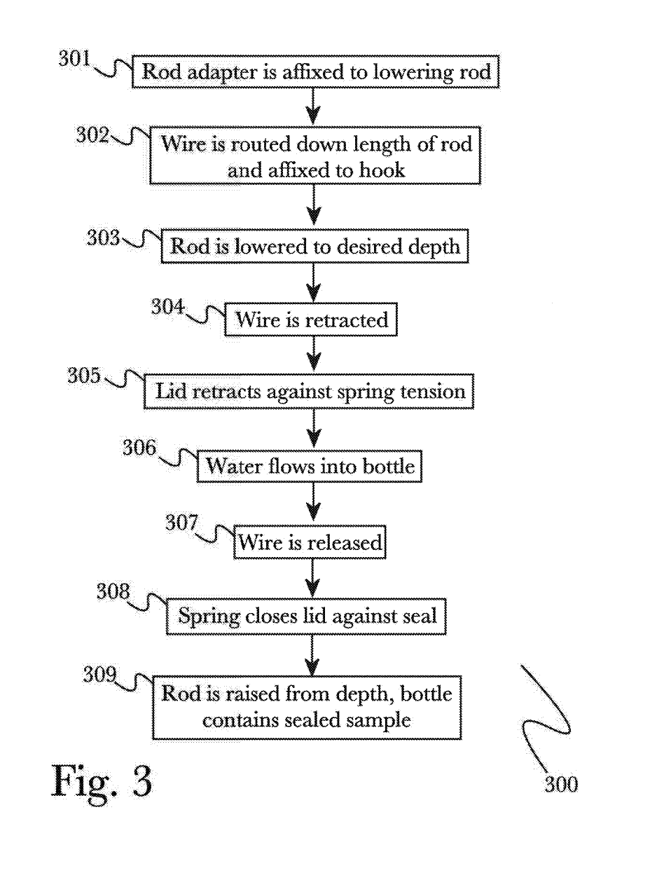

FIG. 3 is an exemplary method diagram 300 according to a preferred embodiment of the invention, illustrating the use 301-309 of a water sampling device 101. Rod adaptor 404, 405 is affixed to lowering rod 301, and a wire or heavy-duty string 108 is routed down length of lowering rod and affixed to an eye hook 106, 302. Rod is lowered to desired depth 303, wire 108 is retracted 304, opening mechanism 110a retracts against spring 104 tension 305. Water sample 202 flows into device opening mechanism 110b canal and into device 101, 306. Wire 108 is released 307, spring 104 closes opening mechanism 110a against opening mechanism 110b forming a seal with O-rings 203a-n, 308. Rod is raised from depth, device 101 contains sealed sample 202, 309.

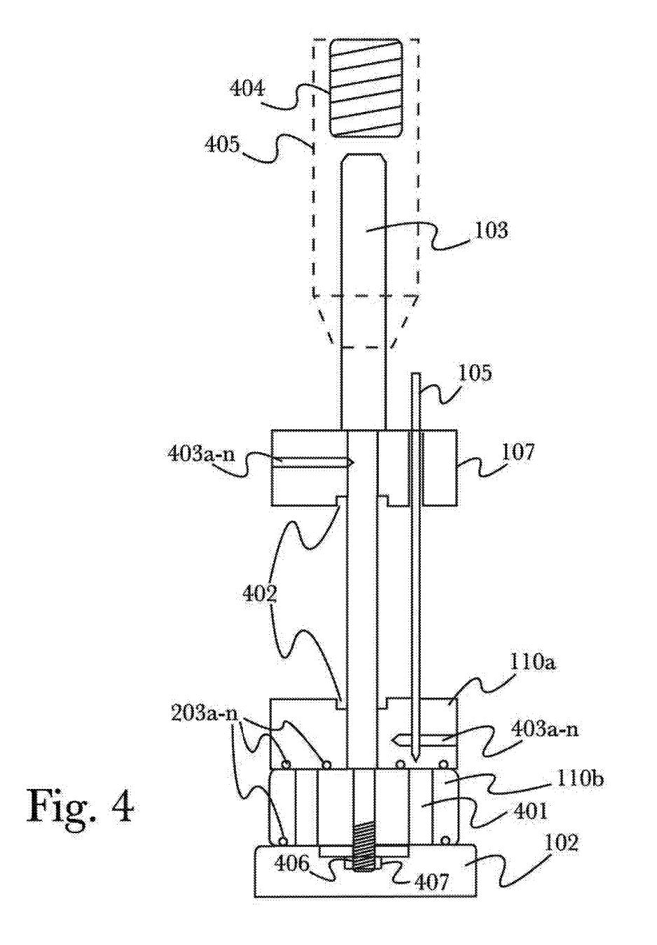

FIG. 4 is an illustration of an exemplary water sampling device mechanisms 400, according to a preferred embodiment of the invention, illustrating all components 401-407 of water sampling device 101 opening mechanisms 110a, 110b. Water sampling device 101 has a threaded lid 102 that securely attaches to device 101. A device connecting rod 103 runs through device disk platform 107, opening mechanism 110a, opening mechanism 110b, and device lid 102 to hold all components to device 101. Device connecting rod 103 securely attaches to device lid 102 by threading 406 onto an acorn nut 407. Attached to device lid 102 with O-rings 203a-n is opening mechanism 110b which has a hollowed out canal 401 for water sample 202 to enter through into device 101. Attached to opening mechanism 110b with O-rings 203a-n is opening mechanism 110a. Opening mechanism 110a has a pin 403a-n attached into it securing lifting rod 105 to opening mechanism 110a. Lifting rod 105 runs up through disk platform 107 which is attached to device connecting rod 103 with a pin 403a-n. Opening mechanism 110a and disk platform 107 have a recess 402 for coiled spring 104 around device connecting rod 103 to rest between. Attached to top of device rod 103 is a threaded 404 rod adaptor 405 for attaching water sampling device 101 to a lowering rod.

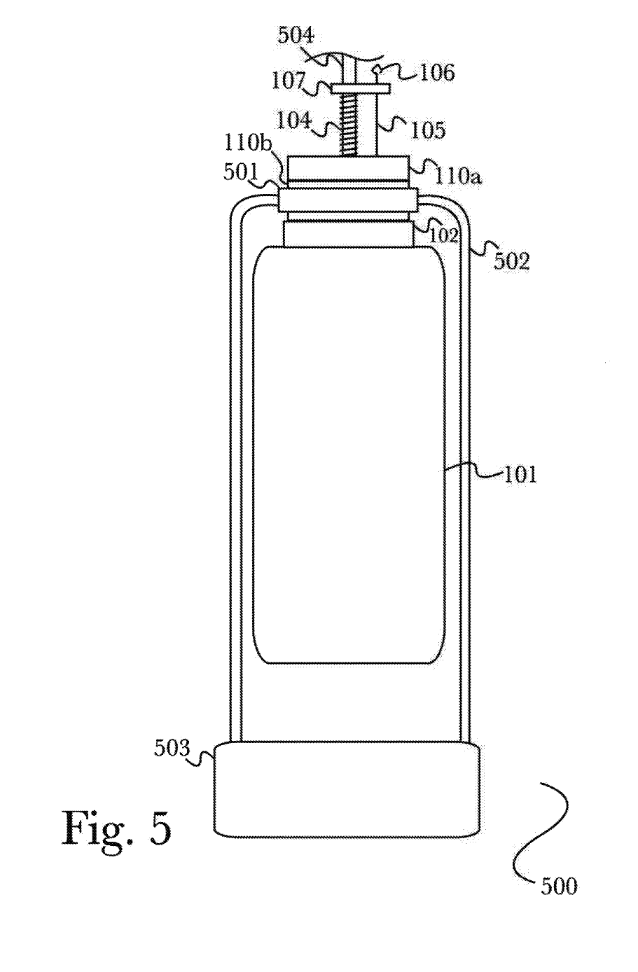

FIG. 5 is an illustration of an exemplary counterweight 500, according to a preferred embodiment of the invention, illustrating an alternative method for lowering water sampling device 101 without an extension rod. Counterweight 503 has two metal rods 502 as sides that attach to a connecting collar 501 that slips over opening mechanism 110b which sits atop device lid 102. Counterweight 503 is located underneath water sampling device 101 and attachment rods 502 run up alongside and outside water sampling device 101, attaching to connecting collar 501 that slips over opening mechanism 110b that has a machined groove. Connecting collar 501 is secured to opening mechanism 110b by set screws that intersect machined groove by tightening up against machined groove. It should be noted that machined groove on opening mechanism 110b, is only on opening mechanism 110b and connecting collar 501 does not intersect device lid 102 or opening mechanism 110a. Device 101 is lowered by rope 504 instead of a lowering rod and counterweight 503 keeps device 101 upright against upward pull. Rope 504 attaches to device connecting rod 103 at disk platform 107. Wire or heavy-duty string 108 runs alongside rope 504 and attaches to hook 106 which in turn is attached to lifting pin 105. Opening mechanism 110a raises against a spring 104 when rope 504 is pulled, allowing sample 202 to enter device 101.

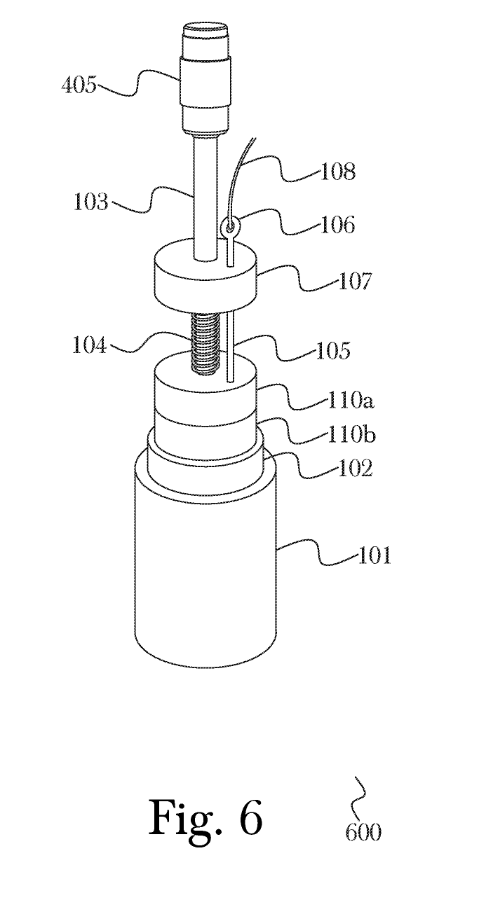

FIG. 6 is an illustration of an exemplary sealed water sampling device, according to a preferred embodiment of the invention, illustrating a three-quarter angle perspective 600 of water sampling device. All components of sealed water sampling device 600 are displayed from a three-quarter angle perspective. Rod adaptor 405 is opened at one end to allow for attachment to a lowering rod, while the other end is affixed to device connecting rod 103. Device connecting rod 103 runs through disk platform 107, opening mechanism 110a, 110b, device lid 102, and device 101. Wrapped around device connecting rod 103 is coiled spring 104, situated between disk platform 107 and opening mechanism 110a. Lifting rod 105 passes through disk platform 107 and attaches to opening mechanism 110a. At one end of lifting rod 105 is eye hook 106 where a wire or heavy-duty string 108 may attach to. Opening mechanism 110a and 110b are attached to airtight lid 102 which is threaded for easier attachment to opening of device 101.

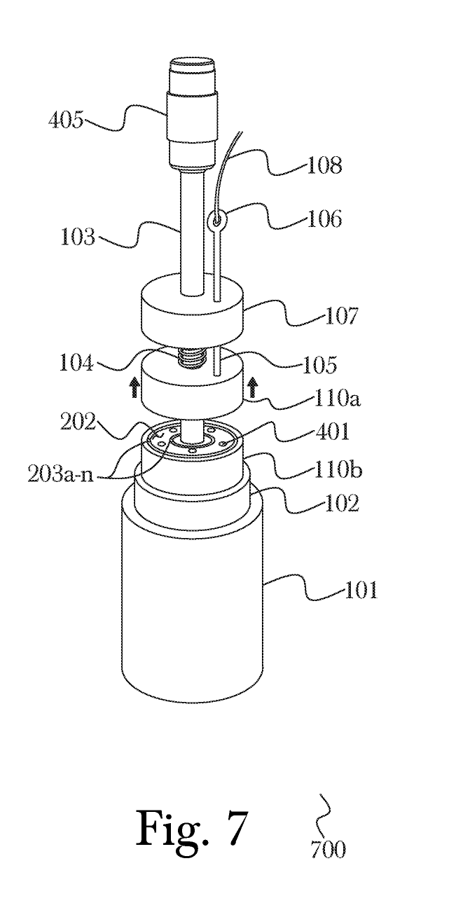

FIG. 7 is an illustration of an exemplary open water sampling device, according to a preferred embodiment of the invention, illustrating a three-quarter angle perspective 700 of water sampling device. All components of opened water sampling device 700 are displayed from a three-quarter angle perspective. Rod adaptor 405 is affixed to device connecting rod 103 which runs through disk platform 107, opening mechanism 110a, 110b, device lid 102, and device 101. Lifting rod 105 runs through disk platform 107 and attaches to opening mechanism 110a; eye hook 106 is located atop lifting rod 105 for wire or heavy-duty string 108 to attach to. Wire or heavy-duty string 108 is pulled upward pulling up lifting rod 105 with it. Lifting rod 105 pulls up opening mechanism 110a against coiled spring 104 (located between opening mechanism 110a and disk platform 107) thereby opening the device 101 to allow water sample 202 entrance to device 101. Water sample 202 enters device 101 through hollow canal 401 located in opening mechanism 110b (which is attached to device lid 102). When lifting rod 105 is lowered, opening mechanism 110a lowers in turn onto O-rings 203a-n located on opening mechanism 110b, forming an airtight seal from the tension of coiled spring 104 against opening mechanism 110b.

The skilled person will be aware of a range of possible modifications of the various embodiments described above. Accordingly, the present invention is defined by the claims and their equivalents.

What is claimed is:

1. An apparatus for a sealed water sampling device, comprising:
a cylindrical-type frame made from a solid material with a narrow mouth and lip where water may enter, with a spring sealed lid to prevent contamination;
wherein at least one end of the device is sealed at all times to prevent contamination of the water sample.

2. The apparatus of claim 1, wherein the opening mechanism is sealed by O-rings and tension from a coiled spring, and the assembled apparatus is attached to a rod adaptor for lowering to a predetermined depth without contamination.
